(19) United States
(12) Patent Application Publication
(10) Pub. No.: US 2007/0083943 A1
(43) Pub. Date: Apr. 12, 2007

(54) Materials and methods for improved sweet corn

(76) Inventors: L. Curtis Hannah, Gainesville, FL (US); Maureen A. Clancy, Gainsville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/577,611

(22) PCT Filed: Nov. 1, 2004
(86) PCT No.: PCT/US04/36266

§ 371 Date: Apr. 28, 2006

Related U.S. Application Data
(60) Provisional application No. 60/516,088, filed on Oct. 31, 2003.

(51) Int. Cl. A01H 5/00; A01H 1/00

(52) U.S. Cl. 800/278; 800/320.1

(57) ABSTRACT

The subject invention pertains to materials and methods for producing improved sweet corn. Sweet corn produced according to the present invention exhibits increased sweetness without a reduction in germination or seedling vigor. One aspect of the invention pertains to a dominant loss-of-function sh2 allele. Another aspect of the invention pertains to a mixture of corn seed that provides for corn plants that produce improved sweet corn. A further aspect of the invention concerns methods for producing corn plants with increased sucrose content in their kernels.

### CROSS-REFERENCE TO RELATED APPLICATION

This application claims the benefit of U.S. Provisional Application Ser. No. 60/516,088, filed Oct. 31, 2003, the disclosure of which is incorporated herein by reference in its entirety.

This invention was made with government support under USDA grant number 9801006. The government has certain rights in the invention.

### BACKGROUND OF THE INVENTION

ADP glucose pyrophosphorylase (AGP) is an important starch biosynthesis enzyme in plants. AGP catalyzes the conversion of ATP and α-glucose-1-phosphate to ADP-glucose and pyrophosphate. ADP-glucose is used as a glycosyl donor in starch biosynthesis by plants and in glycogen biosynthesis by bacteria The importance of ADP-glucose pyrophosphorylase as a key enzyme in the regulation of starch biosynthesis was noted in the study of starch deficient mutants of maize (Zea mays) endosperm (Tsai and Nelson, 1966; Dickinson and Preiss, 1969). Biochemical and genetic evidence has identified AGP as a key enzyme in starch biosynthesis in higher plants and glycogen biosynthesis in E. coli (Preiss et al., 1994; Preiss et al., 1996). AGP catalyzes what is viewed as the initial step in the starch biosynthetic pathway with the product of the reaction being the activated glucosyl donor, ADPglucose. This is utilized by starch synthase for extension of the polysaccharide polymer (reviewed in Hannah 1996).

Initial studies with potato AGP showed that expression in E. coli yielded an enzyme with allosteric and kinetic properties very similar to the native tuber enzyme (Iglesias et al., 1993; Ballicora et al., 1995). Greene et al. (1996a, 1996b) showed the usefulness of the bacterial expression system in their structure-function studies with the potato AGP. Multiple mutations important in mapping allosteric and substrate binding sites have been identified (Okita et al., 1996).

AGP enzymes have been isolated from both bacteria and plants. Bacterial AGP consists of a homotetramer, whereas plant AGP from photosynthetic and non-photosynthetic tissues is a heterotetramer composed of two different subunits. The plant enzyme is encoded by two different genes, with one subunit being larger than the other. This feature has been noted in a number of plants. The AGP subunits in spinach leaf have molecular weights of 54 kDa and 51 kDa, as estimated by SDS-PAGE. Both subunits are immunoreactive with antibody raised against purified AGP from spinach leaves (Copeland et al., 1981; Morell et al., 1988). Immunological analysis using antiserum prepared against the small and large subunits of spinach leaf showed that potato tuber AGP is also encoded by two genes (Okita et al., 1990). The cDNA clones of the two subunits of potato tuber (50 and 51 kDa) have also been isolated and sequenced (Muller-Rober et al., 1990; Nakata et al., 1991). The large subunit of potato tuber AGP is heat stable (Nakata et al., 1991, supra).

As Hannah and Nelson (1975, 1976) postulated, both Shrunken-2 (Sh2) (Bhave et al., 1990) and Brittle-2 (Bt2) (Bae et al., 1990) are structural genes of maize endosperm ADP-glucose pyrophosphorylase. Sh2 and Bt2 encode the large subunit and small subunit of the enzyme, respectively. Based on cDNA sequencing, Sh2 and Bt2 proteins have predicted molecular weight of 57,179 Da (Shaw et al., 1992) and 52,224 Da, respectively. The endosperm is the site of most starch deposition during kernel development in maize. Sh2 and Bt2 maize endosperm mutants have greatly reduced starch levels corresponding to deficient levels of AGP activity. Mutations of either gene have been shown to reduce AGP activity by about 95% (Tsai and Nelson, 1966, supra; Dickinson and Preiss, 1969, supra). Furthermore, it has been observed that enzymatic activities increase with the dosage of functional wild type Sh2 and Bt2 alleles, whereas mutant enzymes have altered kinetic properties. AGP is the rate limiting step in starch biosynthesis in plants. Stark et al. (1992) placed a mutant form of E. coli AGP in potato tuber and obtained a 35% increase in starch content.

The cloning and characterization of the genes encoding the AGP enzyme subunits have been reported for various plants. These include Sh2 cDNA (Bhave et al., 1990, supra), Sh2 genomic DNA (Shaw et al., 1992, supra), and Bt2 cDNA (Bae et al., 1990, supra) from maize; small subunit cDNA (Anderson et al., 1989) and genomic DNA (Anderson et al., 1991) from rice; and small and large subunit cDNAs from spinach leaf (Morell et al., 1988, supra) and potato tuber (Muller-Rober et al., 1990, supra; Nakata et al., 1991, supra). In addition, cDNA clones have been isolated from wheat endosperm and leaf tissue (Olive et al., 1989) and Arabidopsis thaliana leaf (Lin et al., 1988). AGP sequences from barley have also been described in Ainsworth et al. (1995).

AGP has been found to function as an allosteric enzyme in all tissues and organisms investigated to date. The allosteric properties of AGP were first shown to be important in E. coli. A glycogen-overproducing E. coli mutant was isolated and the mutation mapped to the structural gene for AGP, designated as glgC. The mutant E. coli, known as glgC-16, was shown to be more sensitive to the activator, fructose 1,6 bisphosphate, and less sensitive to the inhibitor, cAMP (Preiss 1984). Although plant AGP's are also allosteric, they respond to different effector molecules than bacterial AGP's. In plants, 3-phosphoglyceric acid (3-PGA) functions as an activator while phosphate (PO₄) serves as an inhibitor Dickinson and Preiss, 1969, supra).

There is a great demand for sweeter sweet corn. This is particularly true in Florida where the time from harvest to consumption is several to many days. Traditional sweet corns utilized a mutant recessive allele of the gene termed sugary1 (su1). U.S. Pat. No. 6,410,716 describes isolation of maize sugary1 gene. In the 1960s, John Laughnan at the University of Illinois pointed out the advantages of a recessive mutant allele of shrunken2 (sh2). The mutant sh2 allele conditions two- to four-fold more sugar than does the mutant su1. Corns with the sh2 allele maintain their sweetness (as detected by people) much longer. One consequence of this was the release from Florida of corns like ‘Florida Stay Sweet’.

One problem with traditional sweet corn breeding programs has been that the mutations that result in high sugar and low starch levels are often recessive (thus, plants producing ears destined for consumption must be homozygous for the recessive allele) and, therefore, must be present in both of the inbred lines used to create commercial seed. Hence, breeders have to deal with poor seed germination and seedling vigor in two lines of inbreds. For example, the downside to sh2 “super-sweet corns,” as they are called in the trade, is reduced germination and reduced seedling vigor. Much effort has gone into improvement of these traits by traditional breeding, as well as through the use of a leaky sh2 allele, designated sh2-i U.S. Pat. No. 6,184,438; Giroux and Hannah (1994)), and the use of late promoters to turn the wild type gene on late in kernel development. The mutant sh2-i is so named because it gives an intermediate or leaky phenotype, in which limited AGPase activity is present. Accordingly, starch levels are lower and sucrose levels are higher in the mutant than in a wild-type line. Giroux and Hannah (1994) showed that this particular mutant was caused by a point mutation in the AG dinucleotide at the end of intron 2. This causes slipping of exon 3 during pre-mRNA processing. However, because exon 3 is 123 base pairs in length (a multiple of 3) the reading frame of the mRNA is conserved. It was originally thought that AGPase resulting from the skipping of exon 3 maintained some activity, resulting in the intermediate phenotype. However, it was shown that sites contained in exon 3 are crucial for AGPase function in potato tubers (Greene et al., 1996a). On further analysis, it was found that the skipping of exon 3 in the sh2-i allele only occurs in 90% of the transcripts. In the remaining 10% of transcripts, normal splicing occurs, giving rise to functional AGPase, though in far less concentration compared to wild-type Sh2 alleles (Lal et al., 1999).

### BRIEF SUMMARY OF THE INVENTION

The subject invention pertains to materials and methods for producing improved sweet corn. Sweet corn produced according to the present invention exhibits increased sweetness without a reduction in germination or seedling vigor typically associated with the production of most sweet corns. One aspect of the invention pertains to a dominant loss-of-function (silenced) sh2 allele and plants, plant tissue, and plant seeds having at least one copy of the allele. In an exemplified embodiment, maize having a dominant loss-of-function sh2 allele of the invention was obtained following transformation of maize cells with constructs driven by a Sh2 promoter and containing a polynucleotide encoding a large subunit of AGP that comprises the Rev6 mutation and the heat stable HS33 mutation, and having the Sh1 first intron, and also containing a Nos terminator. Transformants exhibiting the desired characteristics were then identified and further evaluated.

Another aspect of the invention pertains to a mixture of corn seed that provides for corn plants that produce improved sweet corn via increased sucrose content of corn kernels. A further aspect of the invention concerns methods for producing corn plants with increased sucrose content.

### BRIEF DESCRIPTION OF THE DRAWINGS

FIG. 1 shows a comparison of sucrose levels in wild type selfed vs. wild type crossed to Sh2 Silencer. The sharp contrast between a line with a functional Sh2 and a silenced Sh2 is apparent here. Comparative sucrose levels shown for each day post pollination are the average of three ears, except where denoted in Table 2. Numerical data are found in Table 3.

FIG. 2 shows a comparison of sucrose levels in shrunken2-i selfed vs. shrunken2-i crossed to Sh2 Silencer. Silencing effects on an intermediate phenotype is apparent. Data for the sh2iX at 25 dpp is considered spurious since only one ear was harvested. Other points for which three ears were not available are denoted in Table 2. Numerical data are found in Table 3.

FIG. 3 shows a comparison of sucrose levels in sugary selfed vs. sugary crossed to Sh2 Silencer. Comparative sucrose levels shown for each day post pollination are the average of three ears, except where denoted in Table 2. Numerical data are found in Table 3.

FIG. 4 shows a comparison of sucrose levels in shrunken2 selfed vs. shrunken2 crossed to Sh2 Silencer. Comparative sucrose levels shown for each day post pollination are the average of three ears, except where denoted in Table 2. Numerical data are found in Table 3.

### DETAILED DISCLOSURE OF THE INVENTION

The subject invention pertains to materials and methods for producing improved sweet corn. Sweet corn produced according to the present invention exhibits increased sweetness without a reduction in germination or seedling vigor. One aspect of the invention pertains to a dominant loss-of-function (silenced) sh2 allele and plants, plant tissue, and plant seeds having at least one copy of the allele. Another aspect of the invention pertains to a mixture of corn seed that provides for corn plants that produce improved sweet corn. A further aspect of the invention concerns methods for producing improved sweet corn plants having increased sucrose content in their kernels relative to wild type corn or corn that does not have a dominant loss-of function sh2 allele by growing a corn seed mixture of the present invention or by selective planting of corn seed of the invention followed by detasseling of specific corn plants.

In one embodiment of the subject invention, a mixture of corn seed is provided wherein corn seed from a first corn line having a dominant loss-of-function sh2 allele of t-he invention is mixed with corn seed from a second corn line that is genetically male sterile and having a functional or semi-functional sh2 allele or a wild type Sh2 allele. Optionally, the second corn line can contain genes that provide for good eating attributes, such as, for example, a thin pericarp (Abedon et al., 1999). In addition, both the first and second c(-n lines can optionally be homozygous for a recessive sugary (su1) allele and/or contain the sh2-i allele. Corn lines that comprise the su1 allele include the commercially available “Silver Queen,” “Argent,” and “Quickie.” Corn lines that comprise the sh2-i allele are commercially available from Syngenta Seeds (Golden Valley, Minn.). The seed from the first corn line can comprise from about 0.1% to about 50% of the corn seed mixture; thus, the seed from the first corn line can comprise about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% of the corn seed mixture. In one embodiment, the seed from the first corn line can comprise from about 1% to about 50% of the corn seed mixture, or from about 1% to about 25% of the corn seed mixture, or from about 1% to about 15% of the corn seed mixture. In one embodiment, the seed from the first corn line comprises from about 1% to about 10% of the corn seed mixture. In a specific embodiment, the seed from the first corn line makes up about 5% of the seed mix and seed from the second corn line makes up about 95% of the seed mix. The percentage of seed in the mixture can be based on seed weight or seed number.

Because of male sterility, all pollinations would be with pollen from the line having the dominant loss-of-function sh2 allele. Also, because of the dominant nature of the sh2 allele from the first line, all resulting kernels in the pollinated plants will contain high sugar levels. The second line will germinate well if it contains at least one copy of the functional or semi-functional Sh2 allele. The seed that contains the dominant loss-of-function sh2 allele would probably show reduced germination. However, this seed makes up only a small percentage of the total seed mixture. For example, if the seed mix contained 5% first line seed and 95% second line seed, and only 50% of the first line germinated and 98% of the second line germinated, the grower would still garner greater than 95% total germination.

The subject invention also concerns methods of producing corn with increased sucrose content. In one embodiment, a corn seed mix of the present invention is planted and corn plants grown therefrom. The seed of the first corn line contains a dominant loss-of-function sh2 allele. The seed of the second corn line contains a functional or semi-functional Sh2 allele and corn grown from the seed of the second corn line is male sterile or can be made male sterile. Because the second corn line in the methods of the subject invention is male sterile, only the first corn line can act as a pollinator of the second corn line. The second corn line used in the corn mix can be genetically male sterile or can be made male sterile by any suitable method including, but not limited to, mechanical or hand detasseling, or chemical means. In those embodiments where the second corn line is made male sterile by detasseling, those of ordinary skill in the art can readily determine when during growth of the corn plant the tassels should be removed to eliminate pollen shedding. In one embodiment, the second corn line is genetically male sterile. In those embodiments where the second corn line is made male sterile by non-genetic means, then the first and second corn lines must be planted in separate rows so that the two lines can be readily distinguished from each other.

In those embodiments of the subject methods wherein the second corn line is genetically male sterile, the corn seed mix used in the subject method can comprise from about 0.1% to about 50% of the corn seed mixture; thus, the seed from the first corn line can comprise about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 1l%,.12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50of the corn seed mixture. In one embodiment, seed of the first corn line comprises from about 1% to about 50% of the first corn line, or from about 1% to about 25% of the corn seed mixture, or from about 1% to about 15% of the corn seed mixture. In one embodiment, the seed of the first corn line comprises from about 1% to about 10% of the corn seed mixture and the seed of the second corn line comprises from about 99% to 90% of the corn seed mix. In a specific embodiment, the seed from the first corn line makes up about 5% of the seed mix and seed from the second corn line makes up about 95% of the seed mix. Following pollination and maturation of the corn plants grown from the methods of the invention, the corn ears are harvested at a suitable time.

In another embodiment, corn with increased sucrose content can be produced by planting one row of a first corn seed that has a dominant loss-of-function sh2 allele of the present invention, followed by planting one or more rows of a second corn seed that has a functional or semi-functional sh2 or wild type Sh2 allele, then a row of the first corn seed, and then one or more rows of the second corn seed, and so on. After planting, corn is allowed to grow. The tassel on the corn plants grown from the second corn seed is removed prior to pollen shedding. Thus, only pollen from the corn plants having the dominant loss-of-function sh2 allele can pollinate the detasseled corn plants grown from the second corn seed. In one embodiment, 6 to 8 rows of the second corn seed is planted, followed by a single row of the first corn seed, followed by 6 to 8 rows of the second corn seed, and so on in an alternating manner. Optionally, the second corn seed can contain one or more genes that provide for desirable eating attributes or phenotype, such as a thin pericarp. Both the fixst and second corn seed can also optionally be homozygous for a recessive surgary (su1) allele and/or contain the sh2-i allele.

The subject invention also concerns corn plants, plant tissue, and seed comprising a dominant loss-of-function sh2 allele. Corn plants having a dominant loss-of-function sh2 allele can be prepared using standard gene silencing methods, including transcriptional silencing and post-transcriptional gene silencing, to prepare transformed and transgenic plants wherein expression of an Sh2 or functional or semi-functional sh2 gene is silenced. Methods for silencing expression of a targeted gene include, but are not limited to, methods described in U.S. Pat. Nos. 6,573,099 and 6,506,559; in published International Publication Nos. WO 01/01751 and WO 02/044321; and in U.S. Publication Nos. US 2003/0108923 and US 2002/0086356. Constructs comprising a polynucleotide encoding a large subunit of AGP, or a fragment thereof, can be used to transform plant cells. Plants grown from the transformed cells are screened for silencing of shrunken2 expression. In one embodiment, plants with a dominant, loss-of-function sh2 allele were prepared by transforming maize plant cells with sense constructs comprising maize endosperm Sh2 gene containing both a Rev6 mutation (U.S. Pat. Nos. 5,589,618, 5,650,557, and 5,872,216) and an HS 33 mutation (U.S. Pat. Nos. 6,403,863 and 6,069,300), and also comprising the Sh2 promoter, the Sh1 first intron, and the Nos terminator using the protocols described in U.S. application Ser. No. 60/516,088, and described at the “Protocols” hyperlink which can be found at the Worldwide Website: www.agron.iastate.edu/ptf/Web/mainframe.htm#, the disclosure of which is incorporated herein by reference in its entirety. The HS33 mutation conditions a change from a histidine to a tyrosine at position 333 of maize endosperm AGP large subunit amino acid sequence.

Corn seed (designated as “Si1.Sh2”) comprising a dominant loss-of-function sh2 allele was deposited with American Type Culture Collection (ATCC), P.O. Box 1549, Manassas, Va. 20108, on Nov. 1, 2004. The subject corn seed has been deposited under conditions that assure that access to the seed will be available during the pendency of this patent application to one determined by the Commissioner of Patents and Trademarks to be entitled thereto under 37 CFR 1.14 and 35 U.S.C. 122. The deposit will be available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject seed deposit will be stored and made available to the public in accord with the provisions of the Budapest Treaty for the Deposit of Microorganisms, i.e., it will be stored with all the care necessary to keep it viable and uncontaminated for a period of at least five years after the most recent request for the furnishing of a sample of the deposit, and in any case, for a period of at least thirty (30) years after the date of deposit or for the enforceable life of any patent which may issue disclosing the seed. The depositor acknowledges the duty to replace the deposit should the depository be unable to furnish a sample when requested, due to the condition of the deposit. All restrictions on the availability to the public of the subject seed deposit will be irrevocably removed upon the granting of a patent disclosing it.

Corn plants comprising a dominant loss-of-function sh2 allele can be prepared by growing plants from the corn seed of the present invention. The corn plants can then be bred with other corn lines to introduce the dominant loss-of-function sh2 allele into other desirable corn plant genetic backgrounds using standard maize breeding methods. Thus, the subject invention encompasses any corn plant of any genetic background that comprises a dominant, loss-of-function sh2 allele.

The subject invention also concerns corn plants, plant tissue and seeds produced according to any of the methods of the present invention. Optionally, the corn plants, plant tissue, and seeds of the invention are homozygous for a recessive sugary (su1) allele and/or the sh2-i allele. In one embodiment, a corn plant, plant tissue or plant seed comprises one or more genes that provide for desirable eating attributes or phenotype of corn ears.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLE 1

Corn homozygous for the dominant loss-of-function sh2 allele was independently crossed onto four types of corn:

(1) sh2 (standard, null mutant allele that conditions very high levels of sugar);

(2) sh2-i (exhibits great germination, and very high sugars compared to standard sweetcorns, but not as high as the standard, null mutant allele);

(3) sugary (su, standard sweetcorn, i.e., Silver Queen); and

(4) wild type (standard field corn).

Each of the four lines below was also self pollinated. Ear samples were then taken at 14 days, 18 days, and 25 days after pollination. The middle harvest, at 18 days after pollination, is about when people normally eat corn.

Sugar levels in the crossed ears were divided by sugar levels in the selfed ears and the ratios are presented in Table 1 below.

TABLE 1
Fold increase in total sugars conditioned by the silenced Sh2

| | Date post pollination | | | |
|---|---|---|---|---|
| | 14 | 18 | 25 | average |
| sh2 | 0.82 | 1.07 | 1.17 | 1.02 |
| sh2-i | 1.5 | 1.37 | 3.01 | 1.96 |
| sugary | 2.64 | 2.62 | 4.70 | 3.32 |
| wild type | 3.2 | 7.2 | 3.96 | 4.79 |

The Table shows the following: (1) Sugar levels of the silenced Sh2 are comparable to those found in corn having the standard, null, very high sugar allele (sh2), (2) sugar levels in the corn from the cross with the dominant loss-of-function sh2 allele are higher than those in sh2-I especially late in development and (3) the sugar levels in the corn from the cross with the dominant loss-of-function sh2 allele run 2.5 to 7 fold above sugar levels found in wild type and the standard sweet corn, sugary (Silver Queen was used as the sugary source.)

### EXAMPLE 2

Sugar Extraction. Each of the eight genotypes was given a number reference: 1, WT (wild type); 2, WT crossed with the silencer; 3, sh2i; 4, sh2i crossed with the silencer; 5, su; 6, su crossed with the silencer; 7, sh2; 8, sh2 crossed with the silencer. “Wild type” refers to a maize line not lacking any specific gene function and hence it normally synthesizes starch rather than building up sugar. “Silencer” refers to a maize line that contains a dominant silenced (loss-of-function) sh2 allele. Three ears were harvested at 14, 18, and 25 days post pollination (dpp), respectively, for each genotype, with the following exceptions: genotype 8 had no ears at 25 dpp; genotype 4 had only one ear at 25 dpp; genotypes 1 and 2 had only two ears at 14 dpp; genotypes 1, 2, and 7 had only 2 ears at 18 dpp; genotypes 2 and 7 had only two ears at 25 dpp (Table 2). Individual ears of the same genotype that were harvested at the same day post-pollination were given letter designations (A, B, or C). Kernels from each ear were removed in the field and individually stored on dry ice immediately. Long-term storage of kernel tissue was at −80° C.

For extraction, 5 g samples of kernel tissue were ground in liquid nitrogen using mortar and pestle. Samples were suspended in 21 mL of 95% ethanol (heated to 65° C.) in 50 mL Oakridge tubes. Tubes were then covered with large glass marbles, and samples were placed in a 65° C. water bath for 15 minutes. Samples were then centrifuged at 17,000 g for 5 minutes. The supernatants were saved, and pellets were resuspended in 25 mL of 80% ethanol. Again, samples were centrifuged at 17,000 g for 5 minutes, and the supernatants were saved. The supernatants of each sample were then combined and brought to 50 mL with 80% ethanol. For ease of storage, 20 mL of each extract was stored in a scintillation vial at 4° C.

To remove lipids, saved extracts were washed with two subsequent treatments of 10 mL HPLC grade hexane in a 250 mL separation funnel.

Sucrose Determination. Each sample was tested in three trials, using three replicates, giving a total of 27 data points for each genotype at each harvest (with the exception of aforementioned genotypes without three ears). To determine the amount of sucrose in each sample, an adaptation of the Nelson-Somogyi Assay for reducing sugars was used (Somogyi 1952), in conjunction with invertase digestion. In doing so, an undigested replicate could be assayed with a spectrophotometer to measure the reducing sugars in the sample. All sucrose in a sample digested with invertase would be converted into reducing sugar (glucose and fructose), and thereby give a higher reading in the Nelson-Somogyi assay. From the differences in readings between digested and undigested replicates, the amount of sucrose present in the original sample was calculated. For each replicate of a sample, aliquots were diluted in distilled water (ddH₂O) to put concentrations in the measurable range of the assay (0-100 μg/mL).

The first aliquot of each sample was 15 μL and was diluted into 495 μL of H₂O. The second aliquot was 5 μL and was diluted into 495 μL of H₂O and 10 μL of invertase solution (2.0 ng/mL dissolved in ddH₂O). All samples were placed in a water bath at 37° C. overnight to allow complete digestion with invertase. After overnight digestion, 500 μL of each replicate was mixed in a 10 mL glass culture tube with 1.0 mL of the Nelson-Somogyi Copper Reagent. Glass marbles were placed on the culture tubes to reduce evaporative loss, and then the replicates were placed in a boiling water bath for 15 minutes. After cooling to room temperature, 1.0 mL of the Nelson-Somogyi Arsenomolybdate Reagent was mixed with each replicate.

Absorbance readings were then measured in a spectrophotometer at 520 nm. A standard curve was also assayed with glucose standards at 0 μg/mL (ddH₂O), 10 μg/mL, 20 μg/mL, 40 μg/mL, 60 μg/mL, 80 μg/mL, and 100 μg/mL for each set of replicates. Reducing sugar concentrations of replicates were extrapolated from the standard curve using linear regression software found in GraphPad Prism (GraphPad Software, Inc.).

TABLE 2
Number of ears harvested. Attempts were made to harvest 3 ears for
each genotype at each day post-pollination; however, this was not always
possible.

| Assigned | | Days Post-Pollination | Number of Ears |
|---|---|---|---|
| Number | Genotype | at Harvest | Harvested |
| 1 | WT self | 14 | 2 |
| 1 | WT self | 18 | 2 |
| 1 | WT self | 25 | 3 |
| 2 | WTX Sil.Sh2 | 14 | 2 |
| 2 | WTX Sil.Sh2 | 18 | 2 |
| 2 | WTX Sil.Sh2 | 25 | 3 |
| 3 | sh2-i self | 14 | 3 |
| 3 | sh2-i self | 18 | 3 |
| 3 | sh2-i self | 25 | 3 |
| 4 | sh2-iX Sil.Sh2 | 14 | 3 |
| 4 | sh2-iX Sil.Sh2 | 18 | 3 |
| 4 | sh2-iX Sil.Sh2 | 25 | 1 |
| 5 | su self | 14 | 3 |
| 5 | su self | 18 | 3 |
| 5 | su self | 25 | 3 |
| 6 | suX Sil.Sh2 | 14 | 3 |
| 6 | suX Sil.Sh2 | 18 | 3 |
| 6 | suX Sil.Sh2 | 25 | 3 |
| 7 | sh2 self | 14 | 3 |
| 7 | sh2 self | 18 | 2 |
| 7 | sh2 self | 25 | 2 |
| 8 | sh2X Sil.Sh2 | 14 | 3 |
| 8 | sh2X Sil.Sh2 | 18 | 3 |
| 8 | sh2X Sil.Sh2 | 25 | 0 |

Genotype abbreviations indicate the following:
WT, Wild type (Sh2);
sh2-i, shrunken2-I;
su, sugary;
sh2, shrunken2.
Abbreviations followed by an “X” indicated being crossed by silenced shrunken2.

After extrapolation of reducing sugar concentrations, and subsequent calculation of sucrose concentrations, the following averages for each genotype at each day post-pollination were found (Table 3). All concentrations are milligrams of sucrose per gram of fresh kernel tissue.

TABLE 3
Average sucrose concentrations. Average sucrose concentrations for
each genotype at each day post-pollination are given as milligrams sucrose per
gram of fresh kernel tissue. Standard deviations are given in parentheses beneath
each concentration.

| | 1 | 2 | 3 | 4 | 5 | 6 | 7 | |
|---|---|---|---|---|---|---|---|---|
| dpp | (WT) | (WTX) | (sh2-i) | (sh2-iX) | (su) | (suX) | (sh2) | 8 (sh2X) |
| 14 | 4.45 | 36.13 | 31.30 | 40.28 | 14.79 | 39.34 | 46.19 | 51.66 |
| | (4.87) | (1.49) | (9.78) | (4.09) | (3.87) | (7.04) | (3.67) | (7.60) |
| 18 | 13.18 | 46.83 | 28.64 | 48.00 | 9.15 | 58.45 | 66.23 | 39.46 |
| | (3.67) | (10.34) | (3.87) | (12.45) | (3.49) | (3.22) | (4.70) | (7.60) |
| 25 | 2.39 | 31.81 | 23.36 | 74.88 | 6.18 | 37.45 | 35.15 | |
| | (1.39) | (8.31) | (13.41) | (No SD) | (3.11) | (8.10) | (4.69) | |

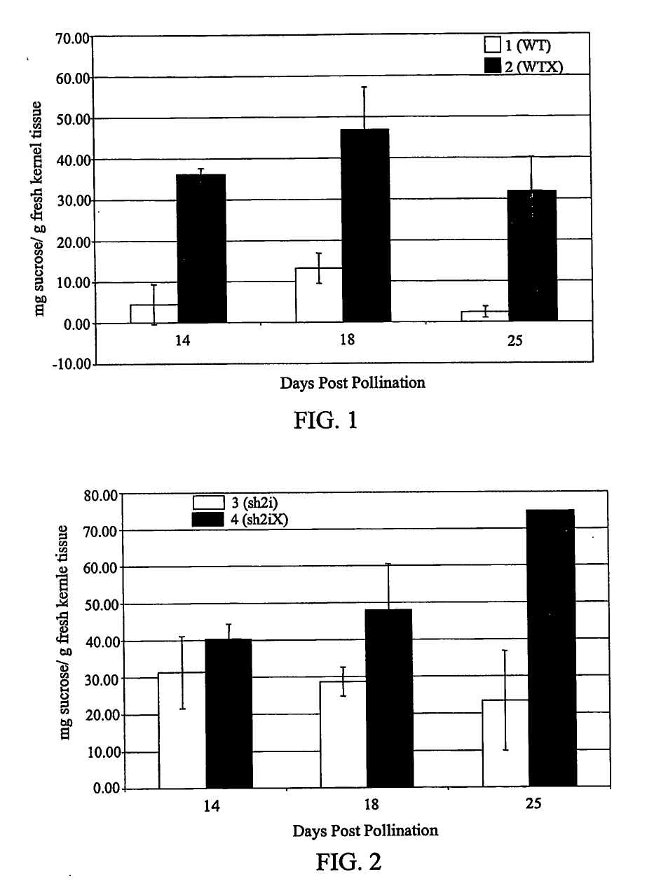

Wild type. The average sucrose concentrations (with standard deviations) for the self-pollinated wild type ears at 14, 18, and 25 dpp were 4.45 (4.87), 13.18 (3.67), and 2.39 (1.39) mg/g, respectively. These are in sharp contrast to the ears resulting from the cross with the silencer, which were 36.13 (1.49), 46.83 (10.34), and 31.81 (8.31) mg/g at 14, 18, and 25 dpp, respectively (FIG. 1).

shrunken2-i. For the self-pollinated shrunken2-i ears, the sucrose concentrations were 31.30 (9.78), 28.64 (3.87), and 23.36 (13.41) mg/g at 14, 18, and 25 dpp, respectively. Crossed ears had sucrose concentrations of 40.28 (4.09), 48.88 (12.45), and 74.88 (no std. dev.) mg/g at 14, 18, and 25 dpp, respectively (FIG. 2).

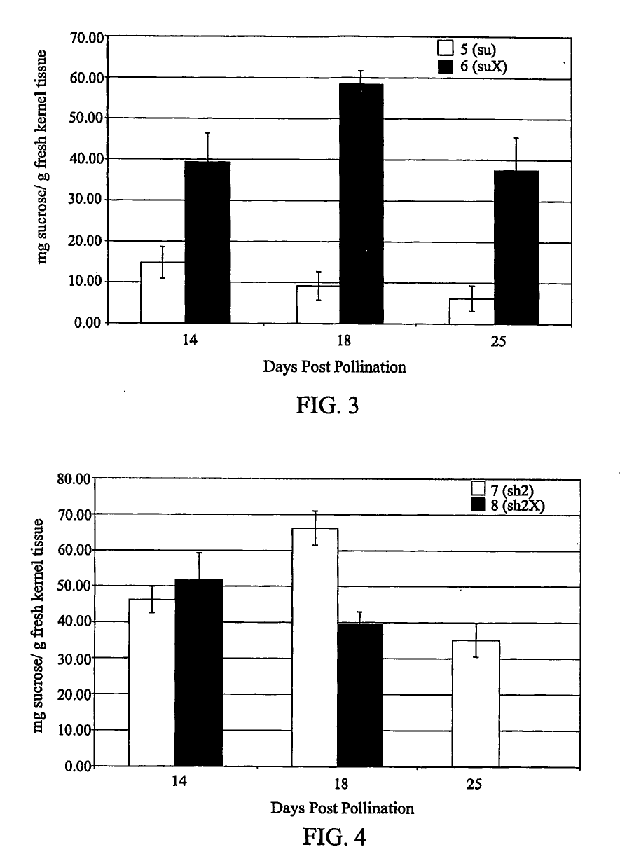

sugary. The sucrose concentration of self-pollinated sugary ears were 14.79 (3.87), 9.15 (3.49), and 6.18 (3.11) mg/g at 14, 18, and 25 dpp, respectively. Ears from sugary crossed with the silencer gave sucrose concentrations at 39.34 (7.04), 58.45 (3.22), and 37.45 (8.10) mg/g at 14, 18, and 25 dpp, respectively (FIG. 3).

shrunken2. Sucrose concentrations of self pollinated shrunken2 ears were 46.19 (3.67), 66.23 (4.70), and 35.15 (4.69) mg/g at 14, 18, and 25 dpp, respectively.

Concentrations of crossed ears were 51.66 (7.60) and 39.46 (3.50) at 14 and 18 dpp, respectively (FIG. 4).

It should be understood that the examples and embodiments described herein are for illustrative purposes only and that various modifications or changes in light thereof will be suggested to persons skilled in the art and are to be included within the spirit and purview of this application and the scope of the appended claims.

### References

U.S. Pat. No. 6,410,716
U.S. Pat. No. 6,184,438
U.S. Pat. No. 6,573,099
U.S. Pat. No. 6,506,559
U.S. Pat. No. 5,589,618
U.S. Pat. No. 5,650,557
U.S. Pat. No. 5,872,216
U.S. Pat. No. 6,403,863
U.S. Pat. No. 6,069,300
WO 01/01751
WO 02/044321
U.S. Publication No. US 2003/0108923
U.S. Publication No. US 2002/0086356
Abedon, B. and Tracy, W. F. (1999) “Ear quality in three sweet corn populations selected for resistance to common rust” J Am Soc Hortic Sci 124:641-643.
Ainsworth, C., Hosein, F., Tarvis, M., Weir, F., Burrell, M., Devos, K. M., Gale, M. D. (1995) “Adenosine Diphosphate Glucose Pyrophosphorylase Genes in Wheat: Differential Expression and Gene Mapping” Planta 197:1-10.
Anderson, J. M., J. Hnilo, R. Larson, T. W. Okita, M. Morell, J. Preiss (1989) “The Encoded Primary Sequence of a Rice Seed ADP-glucose Pyrophosphorylase Subunit and its Homology to the Bacterial Enzyme” J. Biol. Chem. 264:12238-12242.
Anderson, J. M., R. Larson, D. Landencia, W. T. Kim, D. Morrow, T. W. Okita, J. Preiss (1991) “Molecular Characterization of the Gene Encoding a Rice Endosperm-Specific ADPglucose Pyrophosphorylase Subunit and its Developmental Pattern of Transcription” Gene 97:199-205
Armstrong, C. L. and Green, C. E. (1985) “Establishment and maintenance of friable, embryogenic maize callus and the involvement of L-proline” Planta 164: 207-214.
Armstrong, C. L., et al. (1991) “Development and availability of germplasm with high Type II culture formation response” Maize Genetics Coop Newsletter 65: 92-93.
Bae, J. M., M. Giroux, L. C. Hannah (1990) “Cloning And Characterization Of The Brittle-2 Gene Of Maize” Maydica 35:317-322.
Ballicora, M. A., Laughlin, M. J., Fu, Y., Okita, T. W., Barry, G. F., Preiss, J. (1995) “Adenosine 5′-Diphosphate-Glucose Pyrophosphorylase from Potato Tuber” Plant Physiol. 109:245-251.
Bhave, M. R., S. Lawrence, C. Barton, L. C. Hannah (1990) “Identification and Molecular Characterization of Shrunken-2 cDNA Clones of Maize” Plant Cell 2:581-588.
Christensen, A. H. and Quail, P. H. (1996) “Ubiquitin promoter-based vectors from high-level expression of selectable and/or screenable marker genes in monocotylendonous plants” Transgenic Research 5: 213-218.
Chu, C. C., et al. (1975) “Establishment of an efficient medium for anther culture of rice through comparative experiments on the nitrogen sources” Sci. Sinica 18: 659-668.
Copeland, L., J. Preiss (1981) “Purification of Spinach Leaf ADPglucose Pyrophosphorylase” Plant Physiol. 68:996-1001.
Dickinson, D. B., J. Preiss (1969) “Presence of ADP-Glucose Pyrophosphorylase in Shrunken-2 and Brittle-2 Mutants of Maize Endosperm” Plant Physiol. 44:1058-1062.
Frame, B., et al. (2000) “Production of transgenic maize from bombarded Type II callus:

effect of gold particle size and callus morphology on transformation efficiency” In Vitro Cell. Dev. Biol-Plant 36:21-29.
Giroux, M. J., Hannah, L. C. (1994) “ADP-glucose pyrophosphorlyase in shrunken2 and brittle2 mutants of maize” Molecular and General Genetics 243:400-408.
Greene, T. W., Chantler, S. E., Kahn, M. L., Barry, G. F., Preiss, J., Okita, T. W. (1996a) “Mutagenesis of the Potato ADPglucose Pyrophosphorylase and Characterization of an Allosteric Mutant Defective in 3-phosphoglycerate Activation” Proc. Natl. Acad. Sci. 93:1509-1513.
Greene, T. W., Woodbury, R. L., Okita, T. W. (1996b) “Aspartic Acid 413 Is Inportant for the Normal Allosteric Functioning of ADP-Glucose Pyrophosphorylase” Plant Physiol. 112:1315-1320.
Hannah, L. C., O. E. Nelson (1975) “Characterization of Adenosine Diphosphate Glucose Pyrophosphorylases from Developing Maize Seeds” Plant Physiol. 55:297-302.
Hannah, L. C., Nelson, Jr., O. E. (1976) “Characterization of ADP-Glucose Pyrophosphorylase from Shrunken-2 and Brittle-2 Mutants of Maize” Biochem. Genet. 14:547-560.
Hannah, L. Curtis (1996) “Starch synthesis in the maize endosperm,” In: Advances in Cellular and Molecular Biology of Plants, Vol. 4. B. A. Larkins and I. K. Vasil (eds.). Cellular and Molecular Biology of Plant Seed Development. Kluwer Academic Publishers, Dordrecht, The Netherlands.
Iglesias, A., Barry, G.F., Meyer, C., Bloksberg, L., Nakata, P., Greene, T., Laughlin, M. J., Okita, T. W., Kishore, G. M., Preiss, J. (1993) “Expression of the Potato Tuber ADP-glucose Pyrophosphorylase in Escherichia coli” J. Biol Chem. 268:1081-86
Lal, J., Choi, J. H., Hannah, L. C. (1999) “The AG dinucleotide terminating introns is important but not always required for pre-mRNA splicing in the maize endosperm” Plant Physiology 120:65-72.
Lin, T., Caspar, T., Sommerville, C. R., Preiss, J. (1988) “A Starch Deficient Mutant of Arabidopsis thaliana with Low ADPglucose Pyrophosphorylase Activity Lacks One of the Two Subunits of the Enzyme” Plant Physiol. 88:1175-1181.
Morell, M., M. Bloon, V. Knowles, J. Preiss (1988) “Affinity Labeling of the Allosteric Activator Site(s) of Spinach Leaf ADP-glucose Pyrophosphorylase” J. Bio. Chem. 263:633.
Muller-Rober, B. T., J. Kossmann, L. C. Hannah, L. Willmitzer, U. Sounewald (1990) “One of Two Different ADP-glucose Pyrophosphorylase Genes From Potato Responds Strongly to Elevated Levels of Sucrose” Mol. Gen. Genet. 224:136-146.
Murashige, T and Skoog, F. (1962) “A revised medium for rapid growth and bioassays with tobacco tissue cultures” Physiol. Plant. 15: 473-497.
Nakata, P. A., T. W. Greene, J. M. Anderson, B. J. Smith-White, T. W. Okita, J. Preiss (1991) “Comparison of the Primary Sequences of Two Potato Tuber ADP-glucose Pyrophosphorylase Subunits” Plant Mol. Biol. 17:1089-1093.
Okita, T. W., Nakata, P. A., Anderson, J. M., Sowokinos, J., Morell, J., Preiss, J. (1990) “The Subunit Structure of Potato Tuber ADPglucose Pyrophosphorylase” Plant Physiol. 93:785-90.
Okita, T. W., Greene, T. W., Laughlin, M. J., Salamone, P., Woodbury, R., Choi, S., Ito, H., Kavakli, H., Stephens, K. (1996) “Engineering Plant Starches by the Generation of Modified Plant Biosynthetic Enzymes,” In Engineering Crops for Inidustrial End Uses, Shewry, P. R., Napier, S. A., and Davis, P., eds., Portland Press Ltd., London.
Olive, M. R., R. J. Ellis, W. W. Schuch (1989) “Isolation and Nucleotide Sequences of cDNA Clones Encoding ADP-glucose Pyrophosphorylase Polypeptides from Wheat Leaf and Endosperm” Plant Physiol. Mol. Biol. 12:525-538.
Preiss, J. (1984) “Bacterial Glycogen Synthesis and Its Regulation” Ann. Rev. Microbiol. 38:419-458.
Preiss, J. and Romeo, T. (1994) “Molecular Biology and Regulatory Aspects of Glycogen Biosynthesis in Bacteria” Progress in Nuc. Acid Res. and Mol Biol. 47:299-329.
Preiss, J. and Sivak, M. (1996) “Starch synthesis in sinks and sources,” In Photoassimilate distribution in plants and crops: source-sink relationships. Zamski, E., ed., Marcil Dekker Inc. pp. 139-168.
Shaw, J. R., Hannah, L. C. (1992) “Genomic Nucleotide Sequence of a Wild-Type Shrunken-2 Allele of Zea mays” Plant Physiology 98:1214-1216.
Somogyi, M. (1952) “Notes on sugar determination” Journal of Biological Chemistry 195:19-23.
Songstad, D. D., et al. (1996) “Production of transgenic maize plants and progeny by bombardment of Hi II immature embryos” In Vitro Cell. Dev. Biol.-Plant. 32:179-183.
Spencer, T. M., et al., (1990) 37 Bialaphos selection of stable transformants from maize cell culture” Theor. Appl. Genet. 79: 625-631.
Stark et al. (1992) “Regulation of the Amount of Starch in Plant Tissues by ADP Glucose Pyrophosphorylase” Science 258:287-292.
Tsai, C. Y., Nelson, Jr., O. E. (1966) “Starch-Deficient Maize Mutant Lacking Adenosine Diphosphate Glucose Pyrophosphorylase Activity” Science 151:341-343.
Vain, P., et al. (1993) “Osmotic treatment enhances particle bombardment-mediated transient and stable transformation of maize” Plant Cell Reports 12: 84-88.

1. A corn seed mixture comprising
a) corn seed from a first corn line comprising a dominant loss-of-function sh2 allele; and
b) corn seed from a second corn line comprising a functional or semi-functional sh2 or wild type Sh2 allele, wherein said second corn line is a genetically male sterile corn line.

2. The corn seed mixture according to claim 1, wherein said corn seed from said first corn line comprises from about 0.1% to about 50% of said corn seed mixture.

3. The corn seed mixture according to claim 1, wherein said corn seed from said first corn line comprises from about 1% to about 50% of said corn seed mixture.

4. The corn seed mixture according to claim 1, wherein said corn seed from said first corn line comprises from about 1% to about 25% of said corn seed mixture.

5. The corn seed mixture according to claim 1, wherein said corn seed from said first corn line comprises from about 1% to about 15% of said corn seed mixture.

6. The corn seed mixture according to claim 1, wherein said corn seed from said first corn line comprises from about 1% to about 10% of said corn seed mixture.

7. The corn seed mixture according to claim 1, wherein said corn seed from said first corn line comprises about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%,17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%,40%,41%,42%,43%,44%,45%,46%,47%,48%,49%, or 50% of said corn seed mixture.

8. The corn seed mixture according to claim 1, wherein said corn seed from said first corn line comprises about 5% of said corn seed mixture and said corn seed from said second corn line comprises about 95% of said corn seed mixture.

9. The corn seed mixture according to claim 1, wherein said corn seed from said second corn line comprises one or more genes that provide for desirable eating attributes or phenotype.

10. The corn seed mixture according to claim 9, wherein said gene that provides for desirable eating attributes or phenotype is a gene associated with or responsible for thin pericarp phenotype.

11. The corn seed mixture according to claim 1, wherein said first corn line or said second corn line or both said first and second corn line is homozygous for a recessive sugary (su1) allele.

12. The corn seed mixture according to claim 1, wherein said first corn line or said second corn line or both said first and second corn line is homozygous for a sh2-i allele.

13. A method for producing a corn plant having kernels with increased sucrose content, said method comprising planting a corn seed mixture according to claim 1 and growing corn plants from said corn seed mixture and allowing for pollination of said corn plants to occur wherein corn ears produced by said pollinated plants comprise kernels exhibiting increased sucrose content.

14. A method for producing a corn plant having kernels with increased sucrose content, said method comprising planting seed of a first corn line that comprises a dominant loss-of-function sh2 allele, and planting seed of a second corn line that contains a functional or semi-functional sh2 allele or wild type Sh2 allele, wherein said corn seed of said first corn line is planted in a separate row from corn seed of said second corn line, growing corn plants from said planted corn seed, wherein corn plants grown from said seed of said second corn line are made male sterile, and allowing for pollination of said corn plants to occur wherein only the plants grown from the first corn line can act as a pollinator of plants grown from the second corn line, and wherein corn ears produced by said pollinated plants comprise kernels exhibiting increased sucrose content.

15. The method according to claim 14, wherein a corn plant grown from said seed of said second corn line is made male sterile by mechanical or hand detasseling.

16. The method according to claim 14, wherein a corn plant grown from said corn seed of said second corn line is made male sterile by chemical means.

17. The method according to claim 14, wherein six to eight rows of corn seed from said second corn line is planted followed by planting a single row of corn seed from said first corn line, wherein rows of corn seed from said second corn line and a row of corn seed from said first corn line are planted in an alternating fashion.

18. A corn plant, corn plant tissue, or corn plant seed produced by a method comprising planting a corn seed mixture according to claim 1 and growing corn plants from said corn seed mixture and allowing for pollination of said corn plants to occur wherein corn ears produced by said pollinated plants comprise kernels exhibiting increased sucrose content; or
by a method comprising planting seed of a first corn line that comprises a dominant loss-of-function sh2 allele, and planting seed of a second corn line that contains a functional or semi-functional sh2 allele or wild tpe Sh2 allele, wherein said corn seed of said first corn line is planted in a separate row from corn seed of said second corn line, growing corn plants from said planted corn seed, wherein corn plants grown from said seed of said second corn line are made male sterile, and allowing for pollination of said corn plants to occur wherein only the plants grown from the first corn line can act as a pollinator of plants grown from the second corn line, and wherein corn ears produced by said pollenated plants comprise kernels exhibiting increased sucrose content.

19. The corn plant, corn plant tissue, or corn plant seed according to claim 18, wherein said corn plant, corn plant tissue, or corn plant seed comprises a sugary (su1) allele in a homozygous state.

20. The corn plant, corn plant tissue, or corn plant seed according to claim 18, wherein said corn plant, corn plant tissue, or corn plant seed comprises an sh2-i allele in a homozygous state.

21. The corn plant, corn plant tissue, or corn plant seed according to claim 18, wherein said corn plant issue is an ear of corn.

22-30. (canceled)
